SEQUENCE LISTING (8 sequences)

SEQ ID NO: 1 - DNA, 64 bp, Artificial Sequence
  Oligonucleotide encoding for PTB shRNA1.
gatccccaggtgacagccgaagtgcattcaagagatgcacttcggctgtcaccttttttggaaa

SEQ ID NO: 2 - DNA, 64 bp, Artificial Sequence
  Oligonucleotide encoding for PTB shRNA 1.
agcttttccaaaaatgcacttcggctgtcaccttctcttgaaaggtgacagccgaagtgcaggg

SEQ ID NO: 3 - DNA, 66 bp, Artificial Sequence
  Oligonucleotide encoding for shRNA 2.
gatccccaacttccatcattccagagaattcaagagattctctggaatgatggaagtttttggaaa

SEQ ID NO: 4 - DNA, 66 bp, Artificial Sequence
  Oligonucleotide encoding for shRNA 2.
agcttttccaaaaattctctggaatgatggaagtctcttgaaaacttccatcattccagagaaggg

SEQ ID NO: 5 - DNA, 64 bp, Artificial Sequence
  Oligonucleotide encoding for shRNA 3.
gatcccctgacaagagccgtgactacttcaagagagtagtcacggctcttgtcatttttggaaa

SEQ ID NO: 6 - DNA, 64 bp, Artificial Sequence
  Oligonucleotide encoding for shRNA 3.
agcttttccaaaaatgacaagagccgtgactactctcttgaagtagtcacggctcttgtcaggg

SEQ ID NO: 7 - DNA, 20 bp, Artificial Sequence
  PCR primer for amplifying PTB cDNA fragment shown in figure 4B.
ccacctttgccattcctcaa

SEQ ID NO: 8 - DNA, 20 bp, Artificial Sequence
  PCR primer for amplifying the PTB cDNA fragment shown in figure 4B.
aaagaggctttggggtgtga

(19) United States
(12) Patent Application Publication
(10) Pub. No.: US 2009/0325200 A1
(43) Pub. Date: Dec. 31, 2009

(54) POLYPYRIMIDINE-TRACT BINDING PROTEIN (PTB) AS A BIOMARKER AND TARGET FOR THE DIAGNOSIS AND TREATMENT OF CANCER

(76) Inventors: William T. Beck, Chicago, IL (US); Xiaolong He, Palatine, IL (US)

Correspondence Address: POLSINELLI SHUGHART PC, 700 W. 47TH STREET, SUITE 1000, KANSAS CITY, MO, 64112-1802, US

(21) Appl. No.: 12/293,915

(22) PCT Filed: Mar. 23, 2007
(86) PCT No.: PCT/US2007/007352

§ 371 Date: May 1, 2009

Related U.S. Application Data
(60) Provisional application No. 60/785,914, filed on Mar. 24, 2006.

(51) Int. Cl. G01N 33/574; C12N 5/00

(52) U.S. Cl. 435/7.23; 435/377

(57) ABSTRACT

The present invention provides a method of diagnosing cancer in a mammalian subject comprising measuring the level of polypyrimidine-tract binding protein (PTB) in a tissue sample obtained from the subject. An elevated level of PTB in the sample comparing to a normal control is indicative the subject is positively diagnosed with cancer. The present invention furthers provides a method for inhibiting proliferation of a mammalian cancer cell. The method comprises suppressing the level of polypyrimidine-tract binding protein (PTB) in the cell.

### CROSS-REFERENCE TO RELATED APPLICATION

This application claims the priority of United States provisional application Ser. No. 60/785,914 filed Mar. 24, 2006, which is incorporated herein by reference and made a part hereof.

### FEDERALLY SPONSORED RESEARCH OR DEVELOPMENT

This invention was made with United States government support under National Institutes of Health grant number CA27469 awarded to the Gynecologic Oncology Group (GOG) and subcontracted to William T. Beck. The United States government has certain rights in the invention.

### BACKGROUND OF THE INVENTION

1. Field of the Invention

The present invention is related generally to the use of polypyrimidine-tract binding protein (PTB, official gene name: polypyrimidine tract binding protein 1; official gene symbol: PTBP1, and Gene ID: 5725) as a biomarker and target for diagnosis, prognosis and treatment of cancer, particularly ovarian cancer and breast cancer.

2. Background of the Invention

Polypyrimidine-tract binding protein (PTB) is an RNA binding protein with multiple functions in the regulation of RNA processing and internal ribosomal entry site (IRES)-mediated translation. PTB is a member of the heterogeneous nuclear ribonucleoprotein (hnRNP) family (also known as hnRNP I). It was originally identified as a protein that bound to the pyrimidine-rich region within introns of pre-mRNA and was proposed as a splicing factor (Garcia-Blanco et al., 1989; Wang & Pederson, 1990). It is now widely believed that PTB functions as a negative regulator of pre-mRNA splicing, blocking the inclusion of numerous alternative exons into mRNA (Black, 2003; Wagner & Garcia-Blanco, 2001). For example, several highly tissue-specific exons, such as exon 7 of rat β-tropomyosin and the neuron-specific N1 exon of c-src, are repressed for splicing by PTB in all tissues except the restricted cells expressing them. Besides roles in splicing, PTB has also been implicated in the regulation of other aspects of RNA metabolism, such as pre-mRNA polyadenylation (Castelo-Branco et al., 2004; Lou et al., 1996; Lou et al., 1999; Moreira et al., 1998), mRNA stability (Hamilton et al., 2003; Knoch et al., 2004; Kosinski et al., 2003), mRNA export from the nucleus (Zang et al., 2001) and mRNA localization in the cytoplasm (Cote et al., 1999). Moreover, due to its ability to stabilize mRNAs encoding the proteins of pancreatic β-cell secretory granules, thus increasing their translation, PTB is required for the biogenesis of insulin secretory granules (Knoch et al., 2004). In addition, PTB is involved in the control of cap-independent translation driven by the IRES. PTB can bind the IRESs of viral and cellular mRNAs and either positively or negatively influences the IRES activity (Cornelis et al., 2005; Mitchell et al., 2001).

Full-length PTB is composed of four atypical RNA recognition motifs (RRM1)-RRM4) plus a conserved N-terminus carrying a nuclear localization signal and linker peptides between RRMs (Wagner & Garcia-Blanco, 2001). Recent studies on the structure of PTB have revealed that PTB is a monomer in solution and each of four RRM binds RNA with a different specificity. When bound to two separate pyrimidine tracts on the same RNA, PTB induces the formation of a RNA loop (Oberstrass et al., 2005). Several PTB splice variants have been described (Hamilton et al., 2003; Wagner & Garcia-Blanco, 2001; Wollerton et al., 2004). The most common three PTB splice variants differ by the skipping of whole or partial exon 9, which encodes 26 amino acids between RRM2 and RRM3. Partial exon 9 skipping causes a deletion of seven amino acids (Wagner & Garcia-Blanco, 2001). It appears that these three variants are functionally redundant with differences only in the strength of activity (Wollerton et al., 2001). A much smaller PTB variant with exon 3 to exon 9 skipped, thus lacking RRM1 and 2 as well as nuclear localization sequence, was identified in human T lymphocytes and found to be involved in the regulation of CD154 mRNA turnover. It was shown that binding of this small PTB variant to the CD154 3′UTR might be responsible for the instability of CD154 mRNA, which was opposite to the activity of full-length PTB (Hamilton et al., 2003). CD154 is the ligand of CD40 receptor expressed on B cells, macrophages and other antigen-presenting cells, its expression by activated CD4⁺ T cells and its interaction with CD40 is critical in eliciting both humoral and cell-mediated immune responses (Foy et al., 1996).

PTB is widely expressed in many cells and tissues. It is mainly localized to the nucleus and distributed diffusely throughout the nucleoplasm with high concentration in a nuclear structure called the perinucleolar compartment (PNC) (Ghetti et al., 1992; Matera et al., 1995), which is much more prevalent in tumor cells than in normal cells (Huang et al., 1997). Recently, it has been reported that higher PNC prevalence significantly correlates with higher malignancy and poorer prognosis of breast cancer (Kamath et al., 2005). Like other hnRNP proteins, PTB can shuttle between the nucleus and the cytoplasm but its export from the nucleus is not coupled with RNA export, which is in contrast to other hnRNP proteins (Kamath et al., 2001). At present, the mechanism underlying the translocation of PTB is not clear. The phosphorylation at Ser-16 by protein kinase A appears a determinant controlling the distribution of PTB between the nucleus and the cytoplasm (Xie et al., 2003). Among the few extracellular triggers identified so far, glucose can induce the nuclear export of PTB in pancreatic β-cells (Knoch et al., 2004). It is possible that this action is mediated through cAMP-PKA pathway.

We previously observed that human ovarian tumors overexpressed PTB and another splicing factor, SRp20, compared to matched normal ovarian tissues. Correspondingly, we found more splice variants of the multidrug resistance-associated protein 1 (MRP1/ABCC1) as well as CD44 in ovarian tumors than in matched normal ovarian tissues (He et al., 2004). It remains to be determined whether these two splicing factors directly participate in the splicing of the MRP1 and CD44 genes. Others found that overexpression of PTB in glioblastoma tissues coincided with the increased exclusion of the α-exon of fibroblast growth factor receptor 1 (FGFR1) in transformed glial cells (Jin et al., 2003; Jin et al., 2000). Nevertheless, it is unknown whether PTB plays any functional role in tumor progression.

In the present disclosure, we expand our previous observation to more cancer specimens and confirmed the overexpression of PTB in ovarian and breast tumors at the cellular level. We also show that knockdown of PTB expression by vector-based small interfering RNA (siRNA) greatly impairs ovarian and breast tumor cell growth and malignant potential.

These and other aspects and attributes of the present invention will be discussed with reference to the following drawings and accompanying specification.

### BRIEF SUMMARY OF THE INVENTION

The present invention discloses a method for diagnosing cancer in a mammalian subject comprising measuring the level of polypyrimidine-tract binding protein (PTB) in a tissue sample obtained from the subject. An elevated level of PTB in the sample comparing to a normal control is indicative the subject is positively diagnosed with the cancer. In a preferred embodiment, the subject is a human subject. In another embodiment, the cancer is ovarian cancer or breast cancer.

The present invention further provides a method for inhibiting proliferation of a mammalian cancer cell. The method comprises suppressing the level of PTB in the cell. In a preferred embodiment, the cell is a human cancer cell. In an embodiment, the cancer cell is an ovarian cancer cell. In another embodiment, the cancer cell is a breast cancer cell. In yet another embodiment, the suppression of the PTB is accomplished by using small interfering RNA (siRNA) targeting to PTB mRNA.

### BRIEF DESCRIPTION OF THE DRAWINGS

FIG. 1 is a Western Blot analysis of PTB expression in matched ovarian tumor and normal tissues (T=Tumor; N=Normal). Multiple PTB bands are different splice variants of PTB. The expression levels of PTB are quantified as a ratio of PTB to α-actin expression;

FIG. 2 shows examples of homogeneous staining among the three cores of each case in tissue microarray (TMA). Shown is the staining for PTB;

FIG. 3 is an immunohistochemistry staining for PTB and two other splicing factors (SFs), ASF and U2AF65, on human epithelial ovarian cancer (EOC) TMA. Arrows indicate ovarian surface epithelial cells. Note negative PTB staining in normal ovarian epithelial cells. Magnification, 600×;

FIG. 4 shows PTB expression is up-regulated at both protein and mRNA levels in immortalized ovarian surface epithelial (IOSE) cells and ovarian cancer cell lines compared to normal human ovarian surface epithelial (HOSE) cells. FIG. 4A. shows PTB is overexpressed in IOSE cells and ovarian tumor cell lines. Multiple PTB bands are different splice variants of PTB. The expression levels of PTB are quantified as a ratio of PTB to β-actin expression. FIG. 4B shows PTB expression is also up-regulated at mRNA level in IOSE cells and ovarian cancer cell lines compared to normal HOSE;

FIG. 5 shows the knockdown of PTB expression by lentivirus delivered tetracycline (doxycycline, DOX)-inducible siRNA. A. Schematic structure of lentiviral vectors. B. Expression of PTB in stable A2780 cell clones carrying tTR/KRAB-Red and THsiPTB in the presence or absence of DOX. Control was a stable cell clone carrying tTR/KRAB-Red and LV-THM, the parental lentiviral vector of LV-THsiPTB. Multiple PTB bands are different splice variants of PTB. Clone 3 (A2780/PTBsi3) was used in subsequent experiments;

FIG. 6 shows the knockdown of PTB expression suppresses ovarian tumor cell growth. A: MTT assay of cell proliferation. 100 cells per well were seeded in triplicate in 96-well plate and grown with or without DOX for four days before MTT assay. Shown is the average of three separate experiments. B: Cell growth curve. Two separate experiments were done and had similar trend. Shown are the results of one experiment;

FIG. 7 shows the colony formation of tumor cells in soft agar. A: Colonies in the soft agar. B: Quantitation of colonies. Two separate experiments were done and had similar trend. Shown is the result of one experiment;

FIG. 8 is the result of an in vitro invasiveness assay of ovarian tumor cells. Shown is the result of one of three experiments. A: Invasive cells under microscope (40×). B: Quantitation of invasive cells. Invasive cells were counted under microscope with high magnification (150×). Shown are the average cell numbers of three fields. Arrows indicate the invasive cells;

FIG. 9 shows the detection of non-specific interferon response by RT-PCR. It can be seen that there are no differences in the expression of these genes between cells treated with and without DOX, indicating that induction of PTB siRNA does not elicit non-specific interferon response. β-actin is used as a control;

FIG. 10 is an example of immunohistochemical staining for PTB of benign ovarian tumors, borderline/low malignant potential (LMP) tumors and invasive ovarian tumors;

FIG. 11 is an example of immunohistochemical staining for PTB of stage I to stage IV endometrioid and serous ovarian tumors;

FIG. 12 shows the overpression of PTB in progressively transformed HMECs and breast cancer cell line MCF-7; and

FIG. 13 shows that suppression of PTB expression by siRNA inhibits breast tumor cell proliferation. FIG. 13A shows the suppression of PTB expression by vector-based siRNA. Oligonucleotides encoding shRNAs targeting to three different regions of PTB mRNA were cloned into pSuper vector and the resultant plasmids were then introduced into MCF-7 cells by electroportation. PTB expression in MCF-7 transfectants was assayed by western blot. FIG. 13B shows MTT assay of cell proliferation. The parental MCF-7 or transfectants were seeded in 96-well plate at 125, 500 or 2000 cells per well in triplicate and grown for 5 days before MTT assays.

### DETAILED DESCRIPTION OF THE INVENTION

While this invention is susceptible of embodiment in many different forms, there is shown in the drawings, and will be described herein in detail, specific embodiments thereof with the understanding that the present disclosure is to be considered as an exemplification of the principles of the invention and is not intended to limit the invention to the specific embodiments illustrated

We previously observed that human ovarian tumors overexpressed PTB and another splicing factor, SRp20, compared to matched normal ovarian tissues. Correspondingly, we found more splice variants of the multidrug resistance-associated protein 1 (MRP1/ABCC1) as well as CD44 in ovarian tumors than in matched normal ovarian tissues (He et al., 2004). Nevertheless, it is unknown whether PTB plays any functional role in tumor progression.

In the present disclosure, we have for the first time, established that PTB is directly related to malignancy by demonstrating that the level of PTB is elevated in cancer cells and that suppressing the PTB activity inhibits the proliferation of the cancer cells. Thus, PTB is a useful biomarker for the diagnosis and prognosis for cancer, and PTB is also a useful target for treating cancer, such as ovarian cancer and breast cancer shown in the present disclosure.

PTB is an RNA binding protein with multiple functions in the regulation of RNA processing and IRES-mediated translation. The present invention discloses the overexpression of PTB in a majority of epithelial ovarian tumors revealed by immunoblotting and tissue microarray (TMA) staining. By Western Blot, we found that PTB was overexpressed in 17 out of 19 ovarian tumor specimens compared to their matched normal tissues. By TMA staining, we found PTB expression in 38 out of 44 ovarian cancer cases but only in 2 out of 9 normal adjacent tissues. PTB is also overexpressed in SV40 large T antigen (TAg) immortalized ovarian epithelial cells compared to normal human ovarian epithelial cells. By doxycycline-inducible siRNA technology, we found that knockdown of PTB expression in the ovarian tumor cell line A2780 greatly impaired tumor cell proliferation, anchorage-independent growth and in vitro invasiveness. These results indicate that overexpression of PTB is an important component of the multi-step process of tumorigenesis, and might be required for the development and maintenance of epithelial ovarian tumors. Moreover, because of its novel role in tumor cell growth and invasiveness, shown here for the first time, PTB is a novel therapeutic target in the treatment of cancer such as ovarian cancer as disclosed in the examples of the present disclosure.

In the present disclosure, we have extended our previous observation (He et al., 2004), confirming that PTB is overexpressed in a larger series of epithelial ovarian tumors by immunoblot analysis. What is novel here is that we were able to examine PTB expression in epithelial ovarian cancer (EOC) TMAs by immunohistochemistry. Moreover, we also found that PTB expression was up-regulated in immortalized ovarian epithelial cells as well as in ovarian tumor cell lines, compared to normal or untransformed ovarian epithelial cells, another novel observation. Additionally, we demonstrated that knockdown of PTB expression by siRNA impaired the growth of ovarian tumor cells and diminished their malignant potential. Similarly, we were able to show that PTB expression was up-regulated in immortalized and transformed mammary epithelial cells as well as in epithelial breast tumor cells and that knockdown of PTB expressions by siRNA inhibits the proliferation of breast tumor cells. Furthermore, we have demonstrated that the expression of PTB is associated with malignancy of EOC but not with tumor stage (see Example 2). Together, these results indicate that overexpression of PTB is an important component of a multi-step process of tumorigenesis and might be required for the development and maintenance of epithelial tumors such as ovarian tumors and breast tumors demonstrated herein.

Overexpression of PTB in ovarian and breast tumors indicates that PTB is a useful biomarker for the diagnosis and/or prognosis of cancer. Ovarian cancer was selected as one of the examples in the present disclosure because it is the deadliest disease among all gynecological cancers (Jemal et al., 2005). Two facts account for this dismal outcome: one is the absence of early detection marker; the other is inadequacy of present therapy for advanced disease (Ozols et al., 2004). Therefore, to improve patient survival, it is critical to identify new biomarkers for early detection. Given the current absence of any preventable etiologic factors and effective tools for screening, another way to improve patient survival is to discover biomarkers that can lead to a better management of patients after initial diagnosis (Bast et al., 2005).

The results that knockdown of PTB expression causes suppression of tumor cell proliferation, suppression of anchorage-independent growth, and suppression of invasiveness strongly support the notion that PTB is important in maintaining tumor cell growth and malignant potential. To our best knowledge, this is the first report to show such effects. At present, it is not clear what mechanisms mediate these effects. Given the multiple functions of PTB, these effects could be related to changes in alternative splicing, mRNA stability or IRES-driven translation of certain genes. However, current knowledge about the targets of PTB cannot entirely explain our observations; indeed, in some cases, this knowledge appears contrary to our results. For example, it has been reported that PTB can enhance the IRES-mediated translation of two cellular genes: Apaf-1 and p27^{Kip1} (Cho et al., 2005; Mitchell et al., 2001). Apaf-1 is a protein that triggers apoptosis when complexed with cytochrome c, dATP and caspase 9. p27^{Kip1} is a cyclin-dependent kinase inhibitor, negatively regulating cell proliferation. Obviously, the activity of PTB on these two proteins does not match the results in this disclosure. Therefore, there very likely exist some unidentified substrates of PTB whose splice variants are involved in the maintenance of ovarian tumor cell growth and malignancy by PTB. Despite these gaps in our knowledge, our results support the idea that PTB can be used as a therapeutic target for the treatment of cancer, such as ovarian cancer and breast cancer.

The present invention, therefore, provides a method of diagnosing cancer in a mammalian subject comprising measuring the level of PTB in a tissue sample obtained from the subject. An elevated level of PTB in the sample comparing to a normal control is indicative that the subject is positively diagnosed with cancer. In a preferred embodiment, the subject is a human subject. In another embodiment, the cancer is ovarian cancer. In yet another embodiment, the cancer is breast cancer. To our best knowledge, this is the first time that we established the direct role of PTB in tumorigenesis by showing that knockdown of PTB by siRNA causes suppression of tumor cell proliferation, suppression of anchorage-independent growth, and suppression of invasiveness.

In another embodiment, the present invention discloses a method for determining that PTB is a biomarker for diagnosis and prognosis of a cancer in a mammalian subject comprising: (a) providing a cancer cell from a tissue of the subject and (b) measuring the level of PTB in the cell. Determining that PTB is a biomarker for diagnosis and prognosis of the cancer if (a) PTB level is elevated in the cancer cell comparing to a normal control; and (b) suppressing the PTB level in the cancer cell inhibits the proliferation of the cancer cell. The cancer cell can be a cancer cell from a subject that is positively diagnosed with the cancer, or it can be modified from a normal cell to become a cancer cell such as but is not limited to immortalizing or transforming the normal cell such that the modified cell displays cancer cell characteristics or behaviors. These characteristics and behaviors are well known to those skilled in the art and are well documented (see, for example, Mechanisms of Diseases, edited by S. Tomlinson, A. M. Heagerty and A. P. Weetman, Cambridge University Press, 1997, p. 324-327). Examples of these cancer characteristics or behaviors include, but are not limited to, rapid proliferation, anchorage-independent growth, and invasiveness. The normal control can be a matching non-cancer cell from the same tissue of the same subject, or a normal cell from the same type of tissue of a normal subject without the cancer.

“PTB” in the present disclosure includes the full-length PTB (Wagner & Garcia-Blanco, 2001) as well as any splice variants of the molecule such as but not limited to those described by (Hamilton et al., 2003; Wagner & Garcia-Blanco, 2001; Wollerton et al., 2004). The “tissue sample” in the present disclosure includes but is not limited to any cells, organs (or a portion of an organ), blood (including any component of the blood such as plasma, red blood cells, white blood cells, platelets, and the like), and any body fluids such as saliva, urine, tear and the like. The “level of PTB” in the present invention includes both the level of genetic expression of PTB as well as the protein level, which includes an increase in the production of the PTB molecule, or an increase in the activity of PTB molecule as measured by the binding of the PTB molecule to RNA or by the interactions with other molecules. As shown in Example 1, PTB overexpression in epithelial ovarian cancer cells is shown both at the protein level as well as at the mRNA level.

The present invention further provides a method for inhibiting the proliferation of a cancer cell. The method comprises suppressing the level of PTB in the cell. What is meant by “proliferation” in the present disclosure is the rapid reproduction of the cancer cell. Cell proliferation can be assayed using standard assays such as the calorimetric 3-(4,5-dimethylthiazolyl-2)-2,5-diphenyltetrazolium bromide (MTT) assay shown in Examples 1 and 3. The cancer cell can be a cancer cell from a subject that is positively diagnosed with the cancer, or it can be a cell modified from a normal cell to become a cancer cell such as but not limited to immortalizing or transforming the normal cell such that the modified cell displays cancer cell characteristics or behaviors. These characteristics or behaviors are well known to those skilled in the art and are well documented (see, for example, Mechanisms of Diseases, edited by S. Tomlinson, A. M. Heagerty and A. P. Weetman, Cambridge University Press, 1997, p. 324-327). Examples of these cancer characteristics or behaviors include, but are not limited to, rapid proliferation, anchorage-independent growth, and invasiveness. What is meant by “suppressing the level of PTB” in the present disclosure is that either the level of the genetic expression of the PTB is suppressed, or the activity of the PTB molecule is suppressed at the protein level (which includes the suppression of the production of the PTB molecule). Examples of methods for suppressing the expression of PTB include but are not limited to administering small interfering RNA (siRNA) targeting the PTB mRNA or antisense nucleotide sequences targeting the PTB gene, or by implementing any mechanism leading to the down regulation of the PTB gene. siRNA can be administered directly to the cell with an appropriate delivery vehicle (e.g., encapsulating the siRNA molecule in a liposome) or it can be generated within the cell by administering oligonucleotide pairs encoding the siRNA sequences to the cell via a suitable vector. Methods for identifying suitable siRNA sequences are well known to those skilled in the art, and are described in detail, for example, in World Patent Application WO2007/007317. Guidelines and computer programs for identifying effective siRNA sequences can be found at numerous publicly accessible websites, such as http://www.ambion.com/techlib/tb/tb_{—}506.html and http://www.protocol-online.org/prot/Detailed/3210.html. Examples of suppressing the activity of the PTB at the protein level include but are not limited to using antibodies (monoclonal or polyclonal) against PTB, and using inhibitors of PTB which compete with PTB for its binding sites or interfere with its functional domains. Inhibitors of PTB include both small molecule compounds and large molecules such as but are not limited peptides, polypeptides, proteins, glycoproteins, soluble receptors and carbohydrates targeting PTB or its active site. The inhibitors can be natural or synthetic. In a preferred embodiment, the subject is a human subject. In another preferred embodiment, the cancer cell is an ovarian cancer cell. In yet another preferred embodiment, the cancer cell is a breast cancer cell. In yet a further embodiment, the suppression of the PTB is accomplished by using siRNA targeting to PTB mRNA.

### EXAMPLES

### Example 1

### Polypyrimidine-Tract Binding Protein (PTB) is Overexpressed in Ovarian Tumors and its Suppression Impairs Ovarian Tumor Cell Growth and Malignancy

### Cell Lines and Cell Culture Conditions

Normal human ovarian surface epithelial cells (HOSE) were isolated and pooled from four healthy pre-menopausal women undergoing tubal ligations and were cultured for one passage. These procedures were carried out according to the guidelines of the University of British Columbia Ethics Committee, with informed consent of the patients. Line IOSE 398 was derived from HOSE after transduction by SV40 large T antigen and it senesces at about passage 20. Line IOSE 120T was generated from HOSE cells after sequential transduction with SV40 large T antigen and human telomerase reverse transcriptase (hTERT), which can be propagated indefinitely. These cells were grown in media 199 and MCDB105 (1:1) (Sigma), containing 5% fetal bovine serum (FBS) and 50 μg gentamycin/ml. Epithelial ovarian tumor cell lines PA-1, SKOV3, OVCAR8 and A2780 were obtained from ATCC and maintained in DMEM supplemented with 10% FBS, 2 mM L-glutamine and antibiotics.

### Preparation of Tissue Specimens

Nineteen pairs of ovarian tumor and matched normal tissues were obtained from the GOG Tumor Bank (Columbus, Ohio). The tissue specimens were removed during primary cytoreductive surgery and prior to initiation of front-line chemotherapy from patients with advanced ovarian cancer who provided written consent to participate in the GOG Banking Protocol, GOG 136. Following surgery, the specimens were immediately snap-frozen at the GOG participating institutions, shipped to the GOG Tissue Bank on dry ice, and stored at −70° C. until shipment to the GOG Molecular Pharmacology Core Laboratory for testing. Institutional Review Board approval for this component of the project was obtained by University of Illinois Chicago.

### Tissue Microarray (TMA) of EOC

TMA slides were obtained from the GOG Tissue Bank in Columbus, Ohio. The TMA contained 165 randomly distributed 1 mm cores with 150 cancers and 15 adjacent normal ovaries. Among the cancers, there were 48 cases of advanced stage EOC (25 stage III and 23 stage IV) and one case of uterine adenocarcinoma with up to 3 cores for each case (except one case with 6 cores). Cores of adjacent normal ovarian tissue were from 13 patients with ovarian cancer. Of these, 11 cases had one core for each and 2 cases had two cores for each. Cases were from the GOG 136 protocol and all patients provided informed consent to provide specimens for future research.

### Western Blotting

We extracted total proteins from tissues and cell cultures using T-PER Tissue Protein Extraction Reagent (Pierce Biotechnology, Rockford, Ill.) supplemented with protease inhibitor cocktail (2 mM AEBSF, 1 mM EDTA, 130 mM Bestatin, 14 mM E-64, 1 mM Leupeptin and 0.3 mM Aprotinin) (Sigma, St Louis, Mo.). Proteins were separated by SDS-PAGE on a 4-20% gradient gel and then transferred to a nitrocellulose membrane using a semidry electroblotter. The blot was then blocked in 5% milk and probed with monoclonal antibody PTB (Ab-1) (Oncogene Research Products, San Diego, Calif.), Santa Cruz, Calif.), followed by horseradish peroxidase-conjugated donkey anti-mouse IgG. Signals were detected with ECL Western Blotting detection reagents (Amersham Biosciences, Piscataway, N.J.).

### Immunohistochemical Staining of Tissue Microarray

TMA slides were first deparaffinized three times, 5 min each, in xylene and then rehydrated sequentially in 100%, 95% and 80% ethanol two times, respectively, 5 min each. To quench any endogenous peroxidase, TMA slides were incubated in 0.1% H₂O₂ for 30 min. After antigen retrieval in boiling 10 mM sodium citrate for 10 min, the TMAs were blocked in 1.5% normal horse serum for 2 h, followed by incubation overnight with 1:5 diluted primary antibody PTB (Ab-1) (Oncogene Research Products, San Diego, Calif.) and then with biotinylated secondary antibody for 2 h. The antibody binding was detected by Vectastain ABC reagent (Vector Laboratories, Burlingame, Calif.) and visualized with peroxidase substrate 3,3′-diaminobenzidine (DAB) solution. After staining, slides were evaluated by two pathologists.

Preparation of Lentiviruses Carrying Tetracycline-Inducible Expression Cassette of Short-Hairpin RNA (shRNA)

A siRNA can be generated in the cell from a shRNA, which is formed after transcription of its coding sequence. The sequences of three pairs of oligonucleotides encoding for PTB shRNA 1, shRNA 2 and shRNA 3 are as follows:

shRNA 1:

(SEQ ID NO: 1)

5′GATCCCCAGGTGACAGCCGAAGTGCATTCAAGAGATGCACTTCGGCTG
TCACCTTTTTTGGAAA3′
and

(SEQ ID NO: 2)

5′AGCTTTTCCAAAAATGCACTTCGGCTGTCACCTTCTCTTGAAAGGTGA
CAGCCGAAGTGCAGGG3′;
shRNA 2:

(SEQ ID NO: 3)

5′GATCCCCAACTTCCATCATTCCAGAGAATTCAAGAGATTCTCTGGAAT
GATGGAAGTTTTTGGAAA3′
and

(SEQ ID NO: 4)

5′AGCTTTTCCAAAAATTCTCTGGAATGATGGAAGTCTCTTGAAAACTTC
CATCATTCCAGAGAAGGG3′;
shRNA 3:

(SEQ ID NO: 5)

5′GATCCCCTGACAAGAGCCGTGACTACTTCAAGAGAGTAGTCACGGCTC
TTGTCATTTTTGGAAA3′
and

(SEQ ID NO: 6)

5′AGCTTTTCCAAAAATGACAAGAGCCGTGACTACTCTCTTGAAGTAGTC
ACGGCTCTTGTCAGGG3′.

The siRNA sequences targeting to PTB mRNA at nucleotides 231-249, 1710-1730 and 807-825 (relative to start codon), respectively, are in bold. The annealing of the two oligonucleotides generates the DNA fragment with protruding ends compatible with Hind III and Bgl II restriction enzyme sites respectively. The DNA fragment was first cloned into pSuper vector (OligoEngine, Seattle, Wash.) at Hind III and Bgl II sites downstream of H1 promoter and then along with H1 promoter was subcloned into a third generation of lentiviral vector, LV-THM, by replacing the existing H1 promoter in the vector. The resultant lentiviral vector is called LV-THsiPTB. LV-THM harbors both the tetracycline operon (tetO) and H1 promoter within 3′ LTR/SIN region and the GFP gene as a reporter driven by the EF-1α promoter (Wiznerowicz & Trono, 2003). Once integrated into the chromosome, the activities of both the H1 promoter and the EF-1α promoter are regulated by tetracycline (Wiznerowicz & Trono, 2003). Lentiviruses were generated by cotransfection of HEK293T cells with four plasmids: a lentiviral vector plus pMD2.G (expressing envelop protein VSV-G), pMDLg/pRRE (expressing Gag and Pol) and pRSV Rev (expressing Rev). Media were changed 16 h after transfection and harvested 48 h after transfection. Cell debris in the media was removed by centrifugation at 2500 g for 10 min. The titers of lentiviruses in the media were determined by flow cytometry and ranged from 1 to 5×10⁶ transducing units/ml.

Establishment of Stable Cell Lines Expressing Tetracycline-Inducible PTB siRNA

We first established cell lines transduced by lentiviruses LV-tTR KRAB-Red. Briefly, cells were infected with lentiviruses LV-tTR/KRAB-Red at MOI 2-5 for 16 h. After growing for another day, the infected cells were trypsinized, diluted, and about 300 cells were seeded in a 10-cm petri dish and incubated for 7 to 10 days until colonies formed. Twelve isolated cell colonies were picked and transferred to the wells of a 12-well plate. Positive clones were identified by the expression of red fluorescent protein and expanded. We then re-infected these positive cells with lentiviruses LV-THsiPTB and picked a number of isolated colonies to grow in the presence of 1 μg doxycycline (DOX)/ml. Positive cells were identified by the expression of both red fluorescent protein and GFP. The regulation by DOX of siRNA expression in these infected cells was verified by measuring PTB expression by both RT-PCR and Western Blotting.

### Cell Proliferation Assay

Cell proliferation was determined using the calorimetric 3-(4, 5-dimethylthiazolyl-2)-2,5-diphenyltetrazolium bromide (MTT) assay. Briefly, 100 cells per well were seeded in triplicate in a 96-well plate and grown at 37° C., 5% CO₂ for four days. Then, 25 μl of MTT solution (2 mg/ml in PBS) were added to each well. After incubation at 37° C. for 4 h, medium was removed and 100 μl of DMSO were added to each well to dissolve the formazan produced in the cell. Color density at 560 nm was determined using a microplate reader (Molecular Devices, Sunnyvale, Calif.). Cell growth curves were obtained by counting cells grown in 24-well plates using a Coulter Counter (Beckman Coulter, Fullerton, Calif.). For each cell line, 10⁴ cells/well were seeded in a 24-well plate at day 0. Cells in the wells of columns 1 to 6 were trypsinized and counted at day 1 to day 6, respectively. Cells in the top two rows of wells were grown with DOX (2 μg/ml) and cells in the bottom two rows were grown without DOX.

### Soft Agar Colony Formation

Anchorage-independent growth (AIG) of tumor cells was measured by their abilities to form colonies on soft agar. The assay was performed in 60 mm dishes that contained two layers of soft agar. The bottom layer was 0.4% agarose in DMEM with 5% FBS and the top layer was 0.3% agarose plus 5000 cells in DMEM with 5% FBS and with or without DOX (2 μg/ml). Before the assay, each subline was split into two groups with one group grown with DOX and the other without DOX for two days. The cells grown with DOX were mixed with top agar containing DOX at 2 μg/ml. The cells grown without DOX were mixed with top agar without DOX. After the top soft agarose gelled, 1 ml of growth medium with or without DOX was added to each dish. Following incubation at 37° C., 5% CO₂ for two weeks, the dishes were stained with 0.005% crystal violet overnight at 37° C. and the colonies were counted by hand. All assays were done in duplicate.

In vitro Invasiveness Assay

The invasive property of tumor cells was analyzed using CytoSelect™ Cell Invasion Assay kit (Cell Biolabs, Inc., San Diego, Calif.). Before analysis, each subline was split into two groups with one grown with DOX and the other without DOX for two days. Cells were then trypsinized and resuspended in serum free DMEM. 300 μl of cell suspension at 5×10⁵ cells/ml were added to the inner compartment of the ECM-coated culture insert and 500 μl of DMEM, containing 10% FBS, were added to the lower companion-plate well. After incubation for 48 h at 37° C., 5% CO₂, the non-invading cells were removed from the interior of the inserts by wiping with cotton-tipped swabs and the cells that invaded to the lower side of the membrane were stained in the Cell Stain Solution from the kit. After washing in water and drying in air, those cells that invaded into the lower side were counted under a light microscope with a high magnification objective lens.

### Detection of Interferon Response

The nonspecific interferon response to the DOX-induced PTB siRNA was determined by PCR-based examination of relative expression levels of five genes involved in the interferon response with the Interferon Response Detection Kit (System Biosciences, Mountain View, Calif.). These five genes are 2′,5′-oligoadenylate synthetases 1 and 2 (OAS1 and OAS2), which represent a family of interferon-induced proteins implicated in the mechanism of the antiviral action of interferon (Justesen et al., 2000); myxovirus resistance protein 1 (MX1), an important component of the innate host defense against RNA viruses (Suzuki et al., 2004); interferon-induced transmembrane protein 1 (IFITM1), which plays a role in the antiproliferative activity of interferons (Deblandre et al., 1995); and interferon-stimulated transcription factor 3γ (ISGF3γ), a subunit of the interferon-induced signal transduction and transcription activation complex (Aaronson & Horvath, 2002). The isolation of total RNA from A2780 cells and its sublines was done with TRIZOL Reagent (Invitrogen, Carlsbad, Calif.) by following the accompanying protocol. First-strand cDNAs were synthesized with ThermoScript™ RT-PCR System for First-Strand cDNA Synthesis kit (Invitrogen, Carlsbad, Calif.). PCR reactions were set up as instructed in the User Manual of the Interferon Response Detection Kit with Platinum® Taq DNA polymerase (Invitrogen, Carlsbad, Calif.) as the PCR enzyme. The PCR cycling condition was as follows: 94° C. for 3 min, then 30 cycles of 94° C. for 30 sec, 60° C. for 30 sec and 72° C. for 40 sec followed by 1 cycle of 72° C. for 5 min. PCR products were analyzed by agarose electrophoresis.

### Results

### PTB is Overexpressed in Ovarian Tumor Specimens

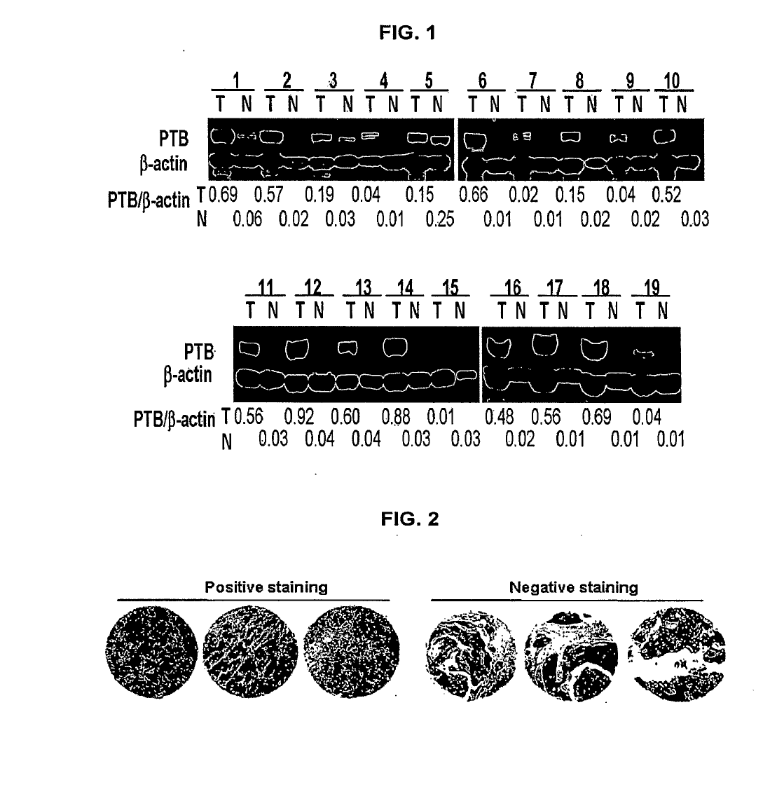

In our previous study, we observed overexpression of PTB in nine out of ten epithelial ovarian tumors compared to their matched normal tissues (He et al., 2004). In order to confirm this observation, we subsequently examined by Western Blotting another 19 pairs of matched ovarian tumor and normal tissues for their expression of PTB. Based on the density ratio of PTB vs β-actin in FIG. 1, PTB is overexpressed in 17 tumors compared to their matched normal tissues. The exceptions are found only in pairs 5 and 15. Among the 19 ovarian tumors, there were 12 serous papillary adenocarcinomas (2, 5, 7-10, 12 and 14-18), 1 mucinous cystadenocarcinoma (4), 1 poorly differentiated endometroid carcinoma (1), 2 clear cell adenocarcinomas (11, 13), 1 cystadenocarcinoma of mixed mullerian epithelial types (3), 1 mixed endometrioid and clear cell carcinoma (6) and 1 Brenner tumor (19).

### Immunohistochemical Staining of TMA for PTB

Of all human ovarian cancers, 85-90% are of epithelial origin (Berek & Bast, 2003). However, in normal ovaries, epithelial cells only account for a very small percentage of total ovarian tissue. Therefore, total normal ovarian tissues are not ideal controls for studying gene expression in epithelial ovarian cancer (EOC). To compare PTB expression at the cellular level between normal ovarian epithelia and ovarian tumors, we performed immunohistochemical staining for PTB on an ovarian tumor TMA that contained tissue spots of 48 cases of advanced epithelial ovarian cancer and 13 cases of normal adjacent ovarian tissues. After staining, 44 cancer cases and 11 normal cases were judged valid and analyzed further. Our rule for valid cases is that there are a minimum of 2 satisfactory cores for cancer and 1 satisfactory core for normal ovary. Unsatisfactory cases are those with missing core(s), scant/insufficient tumor cells, and increased background or folded/wrinkled/torn sections. Overall, the stainings of three cores of a case were very similar, as exemplified in FIG. 2. Out of 44 valid cancer cases, 33 had score differences no greater than 1 both in intensity and in frequency among three cores, indicating that staining was reliable and unbiased.

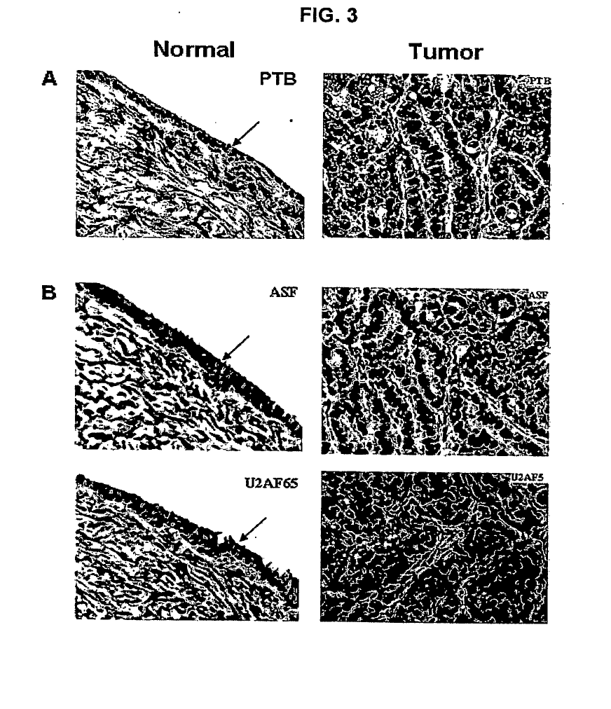

The staining for PTB in the majority of ovarian tumor tissues was positive and strong while in most normal ovarian tissues the staining was negative. FIG. 3A shows examples of PTB staining in tumor and normal ovarian tissues. These staining results are summarized in Table 1.

TABLE 1
Summary of immunohistochemical staining
for PTB in ovarian tumor TMA

| | Negative | Positive | Mixed | p-value * |
|---|---|---|---|---|
| Ovarian Tumor | 6 (13.6%) | 33 (75%) | 5 (11.4%) | <0.0001 |
| Adjacent Normal | 9 (81.8%) | 2 (18.2%) | 0 (0%) | |
| Ovaries | | | | |

It is worth noting that most normal tissues contained only stromal cells, and only one retained surface epithelium. In this case, we found that the staining of normal epithelium for PTB was negative (FIG. 3A), while staining for two other splicing factors, ASF/SF2 and U2AF65, was very strong (FIG. 3B). In contrast, the staining for all three splicing factors was very strong in tumors (FIG. 3A, B). This result suggests that the overexpression of splicing factors in EOC is not universal; indeed, overexpression of PTB in EOC appears to be unique in this disease. In support of this observation, we also stained sixteen conventional normal ovarian tissue sections with some surface epithelia retained and ten epithelial ovarian tumor tissue sections for PTB and confirmed above observation, i.e., the staining of normal ovarian surface epithelia was negative or very weak positive while that of EOC was strong positive. An example of these staining results is shown in FIG. 3C.

### Immortalization of Ovarian Epithelial Cells Increases the Expression of PTB

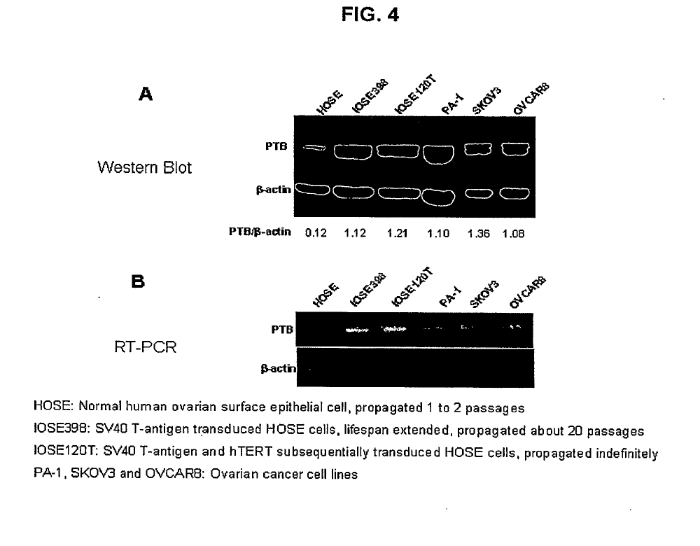

The observation that normal ovarian tissues express low levels of PTB compared to ovarian tumors raises the question of when during oncogenesis this splicing factor becomes overexpressed. Hence, we examined by Western Blotting the expression of PTB in normal HOSE, life-extended HOSE (IOSE398), truly immortalized HOSE (IOSE120T) and ovarian epithelial tumor cell lines PA-1, SKOV3 and OVCAR8. As shown in FIG. 4A, the expression of PTB is substantially overexpressed in life-extended IOSE398 cells and maintained at high levels in IOSE120T and ovarian tumor cells, compared to normal HOSE cells.

FIG. 4B shows that PTB expression is also up-regulated at the mRNA level in immortalized ovarian surface epithelial cells and ovarian cancer cell lines compared to normal HOSE. Total RNAs were extracted from cultured cells with Trizol reagent (Invitrogen, Carlsbad, Calif.) following the manufacturer's instruction. cDNAs were synthesized from 2 μg of total RNA with reagents contained in the ThermoScript RT-PCR system (Invitrogen, Carlsbad, Calif.). The synthesis reactions were run according to the kit manual and incubated at 55° C. for 1 h. The PCR primers for amplifying the PTB cDNA fragment shown in FIG. 4B are CCACCTTTGCCATTCCTCAA (SEQ ID NO:7) and AAAGAGGCTTTGGGGTGTGA (SEQ ID NO:8). The PCR reactions were set up with the reagents contained in ThermoScript RT-PCR system (Invitrogen, Carlsbad, Calif.). The cycling conditions were: 95° C. for 2 min, then 30 cycles of 94° C. for 20 sec, 58° C. for 20 sec, 72° C. for 30 sec min, followed by 1 cycle of 72° C. for 10 min.

This result indicates that the up-regulation of PTB, both at the protein level and at the mRNA level, is an early event in the neoplastic transformation of ovarian epithelial cells.

Knockdown of PTB Expression by Vector-Based siRNA

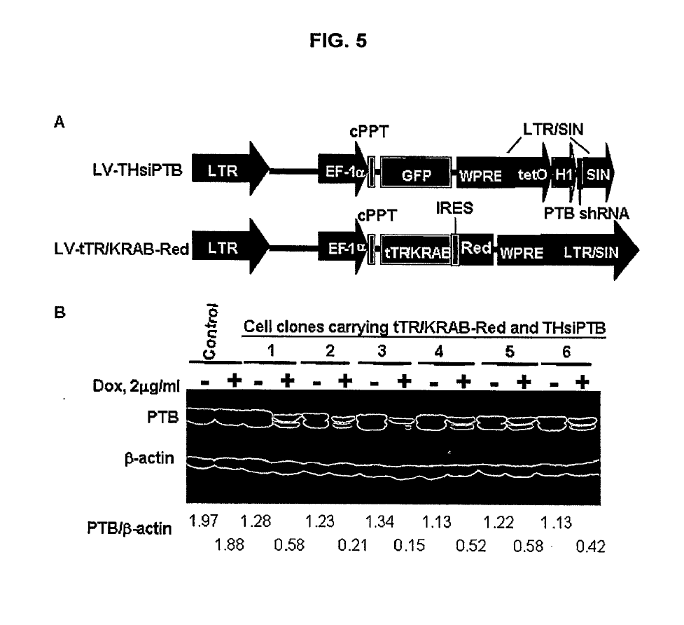

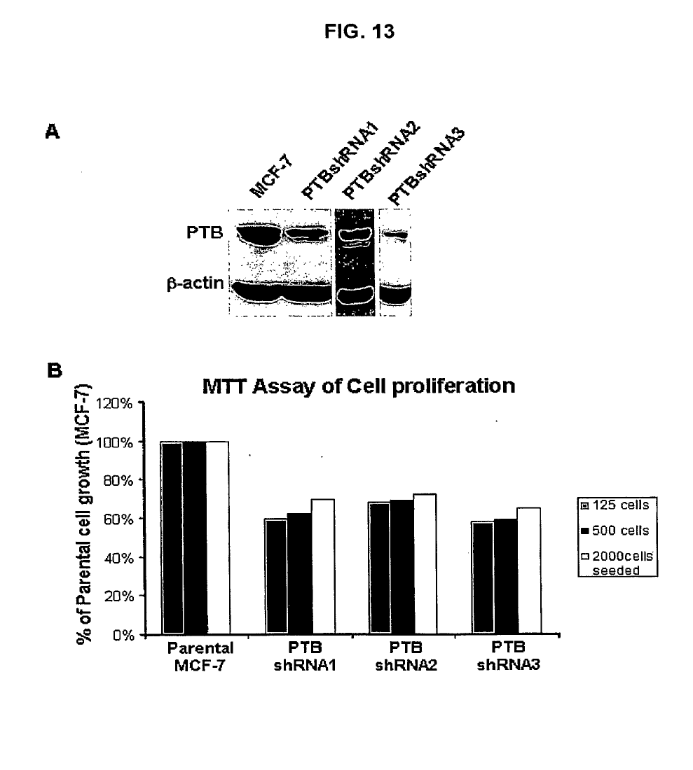

Our above observation raises another question of whether overexpressed PTB plays any functional role(s) in maintaining ovarian tumor cell growth. To address this, we knocked down the expression of PTB in tumor cells and then examined the effects of such manipulation on cell growth and malignancy. In order to significantly knockdown PTB expression, we tested over ten vector-based siRNAs targeting different regions of PTB mRNA and found three to be very effective in suppression of PTB expression, namely shRNA 1, shRNA 2 and shRNA 3 described earlier (FIG. 13A). In this Example, we present the results obtained with shRNA 3. We first established sublines of the epithelial ovarian tumor cell line A2780 that express tetracycline-inducible PTB siRNA. The sublines carry both expression cassettes of tTR/KRAB-Red and PTB shRNA 3 (FIG. 5A). In the presence of DOX, the fusion protein tTR/KRAB-Red is bound by DOX and dissociated from the tetO, thus the downstream PTB shRNA is unblocked and can be expressed. Shown in FIG. 5B is the expression of PTB of six clones in the absence and presence of DOX. It can be seen that the expression of PTB is controlled by DOX. In the presence of DOX, PTB expression is significantly knocked down. In contrast, the expression of PTB in the control subline A2780/LV, which carries LV-tTR/KRAB-Red and the lentiviral vector without PTB shRNA, is not influenced by DOX. Among six clones, according to the comparisons of ratios between PTB and β-actin, clone 3 expresses the lowest levels of PTB after DOX induction of shRNA (PTB expression is decreased nearly 90%). Flow cytometric analysis also revealed that this subline had the highest percentage of GFP-positive cells after repeated passages. Accordingly, the following results are derived from this subline, named as A2780/PTBsi3.

### Knockdown of PTB Expression Suppresses the Growth of Ovarian Tumor Cell In Vitro

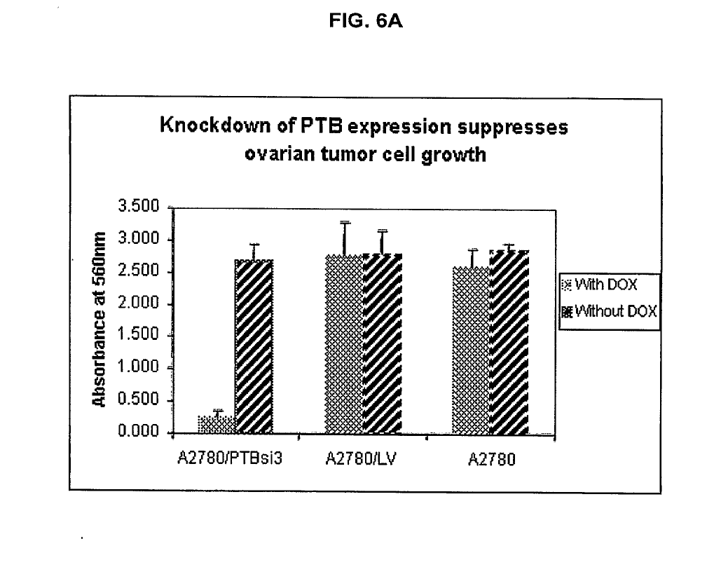

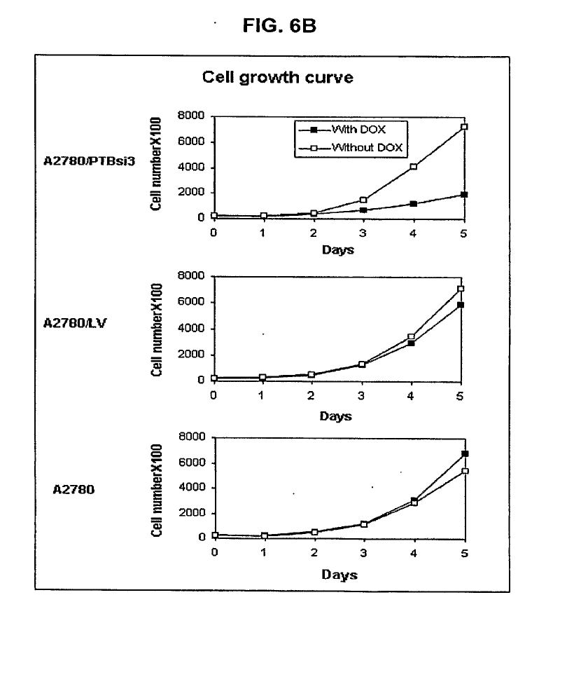

Using the MTT assay, we compared the proliferation of A2780/PTBsi3 in the presence and absence of DOX. As shown in FIG. 6A, the growth of A2780/PTBsi3 was greatly suppressed in the presence of DOX, i.e., when PTB expression was knocked down, while the control subline A2780/LV, and the parental A2780 cells grew similarly in both conditions (with and without DOX). It is worth noting that in the absence of DOX, there is no significant difference in cell growth among the three cell lines, indicating that the introduction of lentiviral vectors into the cell itself did not influence cell growth. We also counted cell numbers at 24 h intervals, using a Coulter Counter (Beckman Coulter, Fullerton, Calif.). The result was highly consistent with that obtained by MTT assay. It can be seen in FIG. 6B that A2780/PTBsi3 grows substantially more slowly in the presence of DOX. By contrast, we observed no such difference in the control subline or parental A2780 cells.

### Knockdown of PTB Expression Lowers Malignant Potential of Ovarian Tumor Cells

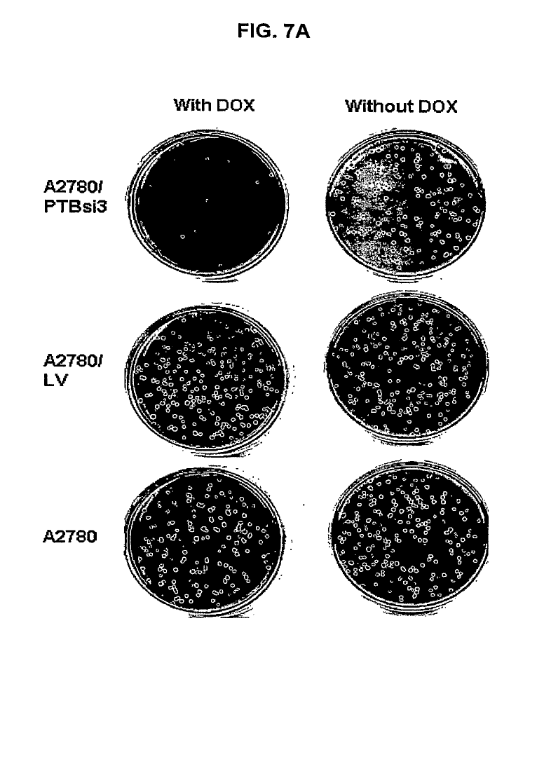

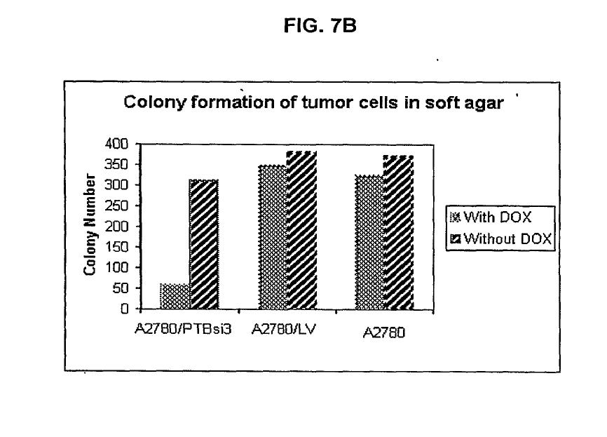

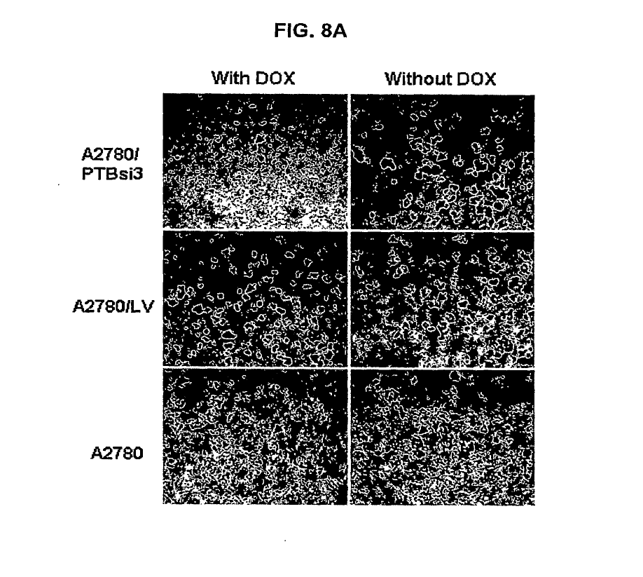

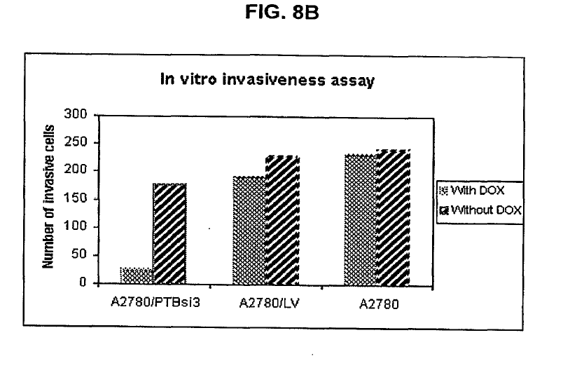

To determine whether overexpression of PTB makes any contribution to the malignancy of ovarian tumor cells, we examined two malignant properties, AIG and invasiveness, of the A2780/PTBsi3 when its PTB expression was manipulated by DOX. The AIG of tumor cells was measured by the formation of colonies in soft agar. As shown in FIG. 7, A2780/PTBsi3 formed 5.4-fold fewer colonies when grown with DOX than without DOX, indicating that knockdown of PTB expression dramatically impaired the AIG of A2780 cells. By contrast, both the control subline and the parental A2780 cells exhibited little or no differences in their ability to form colonies in soft agar when grown with DOX and without DOX. The invasiveness of tumor cells was measured by their abilities to degrade the basement membrane matrix proteins in the coating layer, which serves as a barrier to discriminate invasive cells from non-invasive cells, and ultimately pass through the pores of a polycarbonate membrane as described in the Materials and Methods. As shown in FIG. 8, the A2780/PTBsi3 cells had 7.4-fold lower invasiveness when grown in the presence of DOX than in its absence, indicating that knockdown of PTB expression also greatly reduced invasive potential of A2780 cells. In contrast, DOX treatment produced little or no effects on the invasive abilities of both of the control cell lines.

Knockdown of PTB Expression by siRNA is not Accompanied by Nonspecific Interferon Response

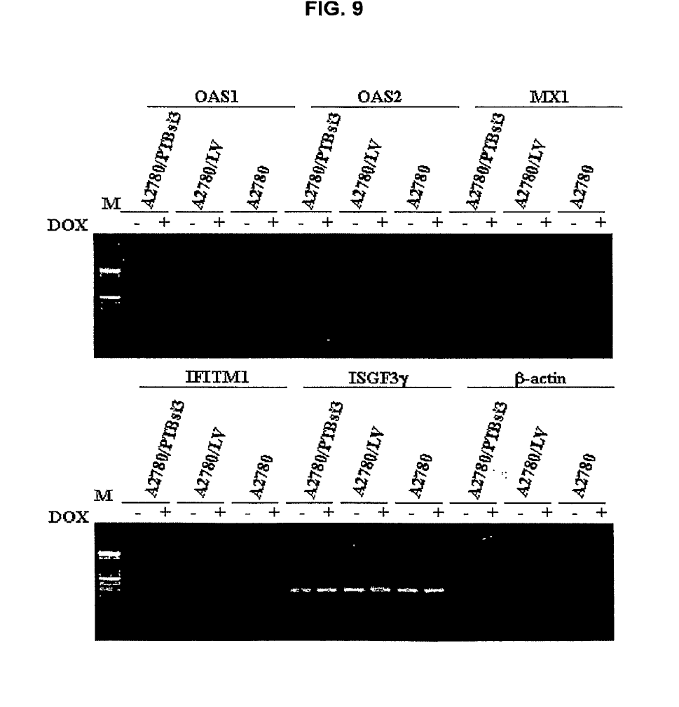

In addition to gene-specific silencing, it has been reported that, some siRNAs, both synthetic and vector-based, can also induce nonspecific effects in cells by activating interferon stimulated genes, which raises a serious concern about specificity of RNA interference and complicates the interpretation of siRNA knockdown results (Bridge et al., 2003; Persengiev et al., 2004; Scacheri et al., 2004). In order to determine whether DOX-induced PTB siRNA elicits nonspecific effects in our cells, we examined by RT-PCR the expression of five genes involved in the interferon response in A2780, A2780/LV and A2780/PTBsi3 grown with or without DOX for 4 days. The nature of these five genes is described in the Materials and Methods and their use helps to ensure the detection of interferon-related stress responses in a variety of cells types and under many different conditions. As shown in FIG. 9, there is no difference in the expression levels of any of the five genes between DOX-treated and DOX-untreated cells, indicating that DOX-induced PTB siRNA does not cause nonspecific interferon responses.

### Example 2

### Expression of PTB is Associated with Malignancy of Epithelial Ovarian Cancer

Based on our observation shown in Example 1 that PTB was up-regulated in epithelial ovarian cancer (EOC), we asked whether the expression of this splicing factor correlates with the malignancy of ovarian tumors and the stages of invasive EOC. As a continuation of our previous study, we examined further, by immunohistochemical staining of EOC tissue microarray (TMA), the expression of PTB in benign, borderline/low malignant potential (LMP) ovarian tumors as well as stage I to stage IV invasive EOC. We found that the expression of PTB is associated with malignancy of EOC but not with tumor stage.

### Tissue Microarray (TMA)

Two types of TMAs were used in this study: one is an ovarian disease progression TMA (G6122) and the other is an ovarian cancer stage TMA (G6121). G6122 contains 174 unique cases of benign tumors, borderline with low malignant potential (LMP) tumors and invasive EOC tissues. The case number for each disease is listed in Table 2. G6121 contains 179 unique cases of stage I to stage IV serous or endometrioid tumors. The case number for each stage is listed in Table 3. All cases in both TMAs were primary tumors obtained after cytoreductive surgery before chemotherapy. These TMAs were created utilizing a 0.6 mm core and each case is represented by three cores, which are distributed randomly on the TMA slide. We obtained the TMA slides from the GOG tumor bank (Columbus, Ohio) and performed the study with the approval of Institutional Review Board of The University of Illinois at Chicago. Patients were consented for donations of specimens on GOG protocol 136 and their identities were not available to us.

TABLE 2
Distribution of cases on ovarian disease progression TMA

| | | | Clear | | |
|---|---|---|---|---|---|
| Disease status | Serous | Mucinous | cell | Peritoneal | Total |
| Benign | 12 | 30 | | | 42 |
| Borderline/LMP | 30 | 30 | | | 60 |
| Invasive | | 30 | 30 | 12 | 72 |
| Total valid cases | 42 | 90 | 30 | 12 | 174 |

TABLE 3
Distribution of cases on ovarian cancer stage TMA

| Stage | Serous | Endometrioid | Total |
|---|---|---|---|
| I | 15 | 27 | 42 |
| II | 23 | 12 | 35 |
| III | 29 | 29 | 58 |
| IV | 30 | 14 | 44 |
| Total | 97 | 82 | 179 |

### Immunohistochemistry Staining

TMA slides were first deparaffinized three times, 5 min each, in xylene and then rehydrated sequentially in 100%, 95% and 80% ethanol two times, respectively, 5 min each. To quench any endogenous peroxidase, slides were incubated in 0.1% H2O2 for 30 min. After antigen retrieval in boiling 10 mM sodium citrate for 10 min, the TMAs were blocked in 1.5% normal horse serum for 2 h, followed by incubation overnight with 1:5 diluted primary antibody PTB (Ab-1) (Oncogene Research Products, San Diego, Calif.) and then with biotinylated secondary antibody for 2 h. The antibody binding was detected by Vectastain ABC reagent (Vector Laboratories, Burlingame, Calif.) and visualized with peroxidase substrate 3,3′-diaminobenzidine (DAB) solution.

### Evaluation of TMA Staining

After staining, the TMA slides were independently evaluated by two pathologists. The evaluation included positiveness of staining, frequency of positive cells and intensity of positive staining. The frequency was scored as 1 to 4 representing positive cells 1% to 25%, 25% to 50%, 50% to 75% and greater than 75%, respectively. The intensity was also scored as 1 to 4 representing weak, definitive, moderately strong and intense staining, respectively.

### Statistical Analysis

SPSS 12 (SPSS Inc., Chicago, Ill.) was used for the data analysis. Pearson's Chi-Square or Fisher's Exact test was performed to obtain the statistical significances of differences between ovarian diseases and between EOC stages.

### Results

### Expression of PTB in Benign, Borderline/LMP Ovarian Tumors and Invasive EOC

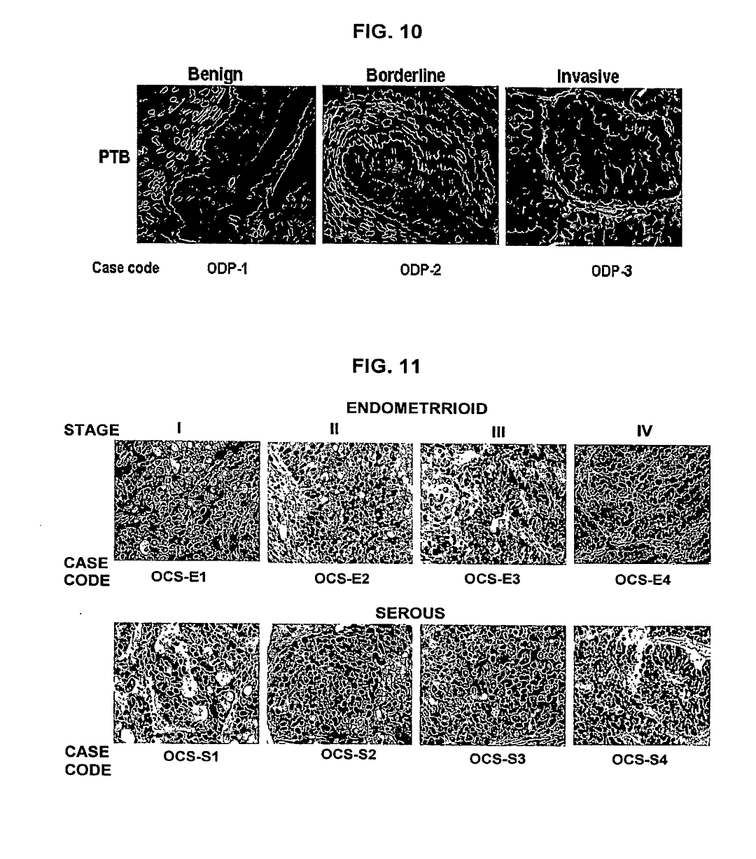

After immunohistochemical staining of G6122 TMA slides for PTB, we had 133 valid cases, and analyzed these further. Our rule for valid cases is that there are a minimum of 2 satisfactory cores for each case. Unsatisfactory cases are those with missing core(s), scant/insufficient tumor cells, increased background or folded/wrinkled/torn sections. Sample staining for PTB in benign, borderline/LMP and invasive EOC are shown in FIG. 10. We categorized the average staining for each case into three groups: negative (at least two cores were negative), positive (at least two cores were positive), and mixed (split negative in one core and positive in one core). Based on this categorization, the result of the staining for PTB is summarized in Table 4. Statistical significance was evaluated using Pearson Chi-Square test. P<0.0001 for overall test including benign, borderline/LMP and invasive tumors. P<0.0001, p=0.0011 and p=0.0034 for pair-wise comparisons between benign and invasive, between benign and borderline/LMP and between borderline/LMP and invasive tumors, respectively.

TABLE 4
Summary of PTB staining in Ovarian Disease Progression TMA

| Disease | Staining | | | | | | | |
|---|---|---|---|---|---|---|---|---|
| status | Negative | | Mixed | | Positive | | Total | |
| Benign | 9 | (52.9%) | 5 | (29.4%) | 3 | (17.6%) | 17 | (100%) |
| Borderline/ | 11 | (22.4%) | 5 | (10.2%) | 33 | (67.3%) | 49 | (100%) |
| LMP | | | | | | | | |
| Invasive | 3 | (4.5%) | 3 | (4.5%) | 61 | (91.0%) | 67 | (100%) |
| Total valid | 23 | | 13 | | 97 | | 133 | |
| cases | | | | | | | | |

As shown in these tables, over 90% of invasive EOC stained positive for PTB, while the majority of benign ovarian tumors stained negative or mixed for PTB. The percentage of borderline/LMP ovarian tumors that were positive was between that of benign and invasive tumors. Statistical analysis of the staining results, using Pearson's Chi-Square test, permits the following conclusions: (1) for overall comparison among benign, borderline/LMP and invasive EOC, the difference in staining for PTB was highly significant (p<0.0001); (2) for pair-wise comparison between benign and invasive EOC, the expression of PTB was significantly lower in benign tumors than in invasive EOC (p<0.001); (3) for pair-wise comparison between borderline/LMP tumors and invasive EOC and between benign and borderline/LMP tumors, the difference in PTB staining was also significant (p<0.01).

Further analysis focusing on mucinous tumors allowed the same conclusions as above for PTB. The staining results in this group of tumors are summarized in Table 5. Statistical significance was evaluated using Pearson Chi-Square test. P values for differences PTB staining: Overall <0.001, benign and LMP <0.05, benign and invasive <0.001, LMP and invasive <0.05. Other subtypes on this TMA cannot be further analyzed because of limited valid cases retained after staining.

TABLE 5
Staining of mucinous ovarian tumors on TMA for PTB

| Disease | Staining | | | | | | | |
|---|---|---|---|---|---|---|---|---|
| status | Negative | | Mixed | | Positive | | Total | |
| Benign | 8 | (53.3%) | 4 | (26.7%) | 3 | (20%) | 15 | (100%) |
| Borderline/ | 11 | (44%) | 1 | (4%) | 13 | (52%) | 25 | (100%) |
| LMP | | | | | | | | |
| Invasive | 3 | (11.5%) | 1 | (3.8%) | 22 | (84.6%) | 26 | (100%) |
| Total | 22 | | 6 | | 38 | | 66 | |

Among cases with at least one positive staining core, we calculated the average frequency of positive ovarian cancer cells. The median value of the average frequency was 3.333 for PTB staining. When the average frequency was categorized into low and high, based on whether it was smaller than the median value, it was associated with ovarian disease status (p<0.01), as shown in Table 6. Regression analysis revealed a moderate relationship between the frequency of PTB staining and disease status (benign, borderline/LMP or invasive) with p=0.047.

TABLE 6
Frequencies of PTB staining in Ovarian Disease Progression TMA

| Disease | Staining | | |
|---|---|---|---|
| status | Low | High | Total |

| | | | | | | |
|---|---|---|---|---|---|---|
| Benign | 7 | (77.8%) | 2 | (22.2%) | 9 | (100%) |
| Borderline/ | 26 | (63.4%) | 15 | (36.6%) | 41 | (100%) |
| LMP | | | | | | |
| Invasive | 23 | (35.4%) | 42 | (64.6%) | 65 | (100%) |
| Total | 56 | (48.7%) | 59 | (51.3%) | 115 | (100%) |

We also calculated the average staining intensity with at least one positive staining core and categorized them as low and high, based on whether they were smaller than the median value. Pearson's Chi-Square test and regression analysis indicated that the average staining intensity for PTB was not associated with or related to the disease status (p=0.167).

In summary, PTB shows differential expression between benign tumors and invasive EOC, between borderline/LMP tumors and invasive EOC, and between benign and LMP tumors.

### Expression of PTB in Stages I to IV Human EOC.

After staining of the G6121 TMA slides for PTB, there were 168 cases that were judged to be valid and analyzed further. The rule for valid cases was the same as above. There were two subtypes of EOC on this TMA: serous and endometrioid. Sample staining of stages I to IV serous and endometrioid EOC for PTB is shown in FIG. 11. The results of average staining, categorized as negative, positive and mixed, are summarized in Table 7.

TABLE 7
Summary of average PTB staining in ovarian cancer stage TMA

| | Staining | | | | | | | |
|---|---|---|---|---|---|---|---|---|
| Stage | Negative | | Mixed | | Positive | | Total | |
| I | | | 5 | (10.9%) | 41 | (89.1%) | 46 | (100%) |
| II | | | 2 | (6.9%) | 27 | (93.1%) | 29 | (100%) |
| III | 1 | (1.8%) | 2 | (3.5%) | 54 | (94.7%) | 57 | (100%) |
| IV | | | 3 | (8.3%) | 33 | (91.7%) | 36 | (100%) |
| Total | 1 | (0.6%) | 12 | (7.1%) | 155 | (92.3%) | 168 | (100%) |

Chi-Square test: p = 0.669

As seen in the figure and shown in the table, almost all cases were stained positive for PTB, which was consistent with what we observed in staining of the G6122 TMA described above. Statistical analysis indicated that there were no significant differences in average staining or frequency of positive cancer cells for PTB among the four stages or between any two stages. However, the average intensity of staining for PTB, when categorized as low and high, based on whether it was smaller than the median value, as shown in Table 8, was associated with categorized stages, i.e. early stage, which included stages I and II, and advanced stage, which included stages III and IV, with greater intensity in the latter cases (p=0.019).

TABLE 8
Average intensity of staining for PTB in ovarian cancer stage TMA

| | Staining | | | | | |
|---|---|---|---|---|---|---|
| Categorized stage | Low | | High | | Total | |
| Early stage (I + II) | 41 | (54.7%) | 34 | (45.3%) | 75 | (100%) |
| Advanced stage (III + IV) | 33 | (35.5%) | 60 | (64.5%) | 93 | (100%) |
| Total | 74 | (44.0%) | 94 | (56.0%) | 168 | (100%) |

Statistical significance was evaluated using Fisher's Exact test. p = 0.019

In this Example, we examined the expression of PTB in ovarian tumors by immunohistochemical staining of two specialized ovarian tumor TMAs, one focusing on tumor progression and the other focusing on cancer stages. Our results show that PTB is differentially expressed among benign ovarian tumors, borderline/LMP ovarian tumors and invasive EOC with benign tumors mostly stained negative, more than 90% of invasive EOC stained positive and borderline/LMP tumors stained between benign and invasive tumors, but no significant differences were found among invasive EOC of different stages, indicating that the expression of PTB is associated with malignancy of ovarian tumors. These results are consistent with our previous observations in vitro and support the notion that overexpression of certain splicing factors, PTB in particular, is probably an early event in the process of ovarian tumorigenesis and is required for maintenance of the malignant properties of ovarian tumor cells. Moreover, the results shown here provide further evidence to suggest that PTB is a useful biomarker for diagnosis of invasive EOC.

Aberrant alternative splicing is a very: common phenomenon found in human tumors (41). Our results herein and those of others (Watermann D. O. et al., 2006) suggest that abnormal regulation of splicing factor expression may make a major contribution to such aberration. Given the importance of alternative splicing in the generation of proteomic complexity, it is conceivable that up- or down-regulation of splicing factors may be an indispensable component of the process of tumorigenesis that is involved in mediating the effects of transformation. Thus, controlling splicing factor expression may turn out to be a very effective way to inhibit tumor cell growth; that is, certain splicing factors may be good therapeutic targets. Our recent in vitro studies in an ovarian tumor cell line, shown in Example 1, support this hypothesis.

### Example 3

### Overexpression of PTB and Effect of Suppressing PTB in Breast Tumor Cells

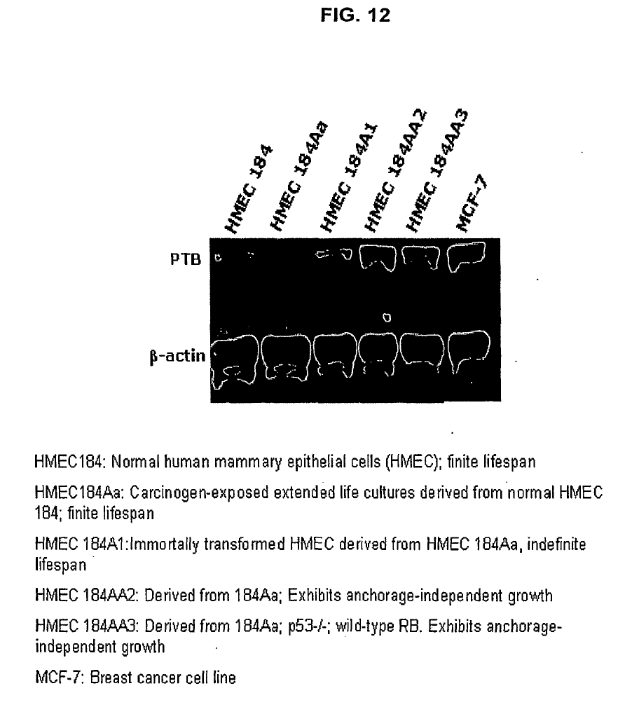

In addition to ovarian cancer cells, we have also examined the overexpression of PTB in another cancer cell, namely the breast cancer cell, and the effect of suppression of PTB expression on breast tumor cell proliferation. The results of these studies are shown in FIGS. 12 and 13.

### Cell Lines and Cell Culture Conditions

Normal human mammary epithelial cell (HMEC 184) and its derivative cell cultures HMEC 184Aa, 184A1, 184AA2 and 184AA3 were gift from Dr. Martha Stampfer of Lawrence Berkeley National Laboratory, see http://www.lbl.gov/˜mrgs/mreview.htm. These cell cultures were grown in MEBM medium (Cambrex Corporate, East Rutherford, N.J.) supplemented with EGF (5 ng/ml), insulin (5 μg/ml), bovine pituitary extract (70 μg/ml), transferrin (5 μg/ml), hydrocortisone (0.5 μg/ml) and isoproterenol (10⁻⁵ M). The MCF-7 cell was purchased from ATCC and was maintained in DMEM supplemented with 10% FBS, 2 mM L-glutamine and antibiotics.

### Western Blotting

We extracted total proteins from cell cultures using T-PER Tissue Protein Extraction Reagent (Pierce Biotechnology, Rockford, Ill.) supplemented with protease inhibitor cocktail (2 mM AEBSF, 1 mM EDTA, 130 mM Bestatin, 14 mM E-64, 1 mM Leupeptin and 0.3 mM Aprotinin) (Sigma, St Louis, Mo.). Proteins were separated by SDS-PAGE on a 10% gel and then transferred to a nitrocellulose membrane using a semidry electroblotter. The blot was then blocked in 5% milk and probed with monoclonal antibody PTB (Ab-1) (Oncogene Research Products, San Diego, Calif.), followed by horseradish peroxidase-conjugated donkey anti-mouse IgG. Signals were detected with ECL Western Blotting detection reagents (Amersham Biosciences, Piscataway, N.J.).

### Transfection of MCF-7 Cells

MCF-7 cells were transfected by electroporation. Briefly, after trypsinization, centrifuge and washing with PBS, the cells were resuspended at 1×10⁷ cells/ml in fresh DMEM without FBS and chilled on ice for 5 minutes. Then 1×10⁶ cells mixed with 50 ug of plasmids were added to a pre-chilled cuvette and pulsed in Bio-Rad Gene Pulser at 25 uF and 500V.

### Cell Proliferation Assay

Cell proliferation was determined using MTT assay. Briefly, cells were seeded in 96-well plate at 125, 500 and 2000 cells per well in triplicate and grown at 37° C., 5% CO2 for five days. Then, 25 μl of MTT solution (2 mg/ml in PBS) were added to each well. After incubation at 37° C. for 4 h, medium was removed and 100 μl of DMSO were added to each well to dissolve the formazan produced in the cell. Color density at 560 nm was determined using a microplate reader (Molecular Devices, Sunnyvale, Calif.)

### Results

As shown in FIG. 12. PTB is overexpressed in progressively transformed human mammary epithelial cells (HMECs) and a breast cancer cell line. PTB is overexpressed in HMEC 184A1 (immortally transformed HMEC derived from HMEC 184a with indefinite lifespan), HMEC 184AA2 (which is derived from 184Aa and exhibits anchorage-independent growth), HMEC 184AA3 (which is derived from 184Aa; p53−/−; wide type Rb, and exhibits anchorage independent growth), and the breast cancer cell line MCF-7, as compared to non-cancer cells including HMEC 184 (normal HMECs with finite lifespan) and HMEC184a (carcinogen-exposed extended life cultures derived from normal HMEC 184 with finite lifespan).

FIG. 13 shows that suppression of PTB expression by siRNA inhibits breast tumor cell proliferation. FIG. 13A shows the suppression of PTB expression by vector-based siRNA. Oligonucleotides encoding siRNAs targeting three different regions of PTB mRNA as described in Example 1 were cloned into pSuper vector and the resultant plasmids were then introduced into MCF-7 cells by electroportation. PTB expression in MCF-7 transfectants was assayed by western blot. FIG. 13B is the effect of suppression of PTB by PTB siRNA on cell proliferation of MCF-7 cells. The parental MCF-7 or transfectants were seeded in 96-well plate at 125, 500 or 2000 cells per well in triplicate and grown for 5 days before MTT assays. As shown in FIG. 13B, suppression of PTB by siRNA inhibits the proliferation of MCF-7 cells.

The practice of the present invention will employ and incorporate, unless otherwise indicated, conventional techniques of cell biology, cell culture, molecular biology, microbiology, genetic engineering, and immunology, which are within the skill of the art. While the present invention is described in connection with what is presently considered to be the most practical and preferred embodiments, it should be appreciated that the invention is not limited to the disclosed embodiments, and is intended to cover various modifications and equivalent arrangements included within the spirit and scope of the claims. Modifications and variations in the present invention may be made without departing from the novel aspects of the invention as defined in the claims. The appended claims should be construed broadly and in a manner consistent with the spirit and the scope of the invention herein.

### REFERENCES

Aaronson D S and Horvath C M. (2002). A road map for those who don't know JAK-STAT. Science, 296: 1653-5.
Bartel F, Taubert H and Harris L C. (2002). Alternative and aberrant splicing of MDM2 mRNA in human cancer. Cancer Cell, 2: 9-15.
Bast R C, Jr., Lilja H, Urban N, Rimm D L, Fritsche H, Gray J, et al. (2005). Translational crossroads for biomarkers. Clin Cancer Res, 11: 6103-8.
Berek J S and Bast R C, Jr. (2003). Ovarian Cancer. BC Decker Inc: Hamilton.
Black D L. (2003). Mechanisms of alternative pre-messenger RNA splicing. Annu Rev Biochem, 72: 291-336.
Bridge A J, Pebernard S, Ducraux A, Nicoulaz A L and Iggo R. (2003). Induction of an interferon response by RNAi vectors in mammalian cells. Nat Genet, 34: 263-4.
Bryan T M and Reddel R R. (1994). SV40-induced immortalization of human cells. Crit. Rev Oncog, 5: 331-57.
Caceres J F and Kornblihtt A R. (2002). Alternative splicing: multiple control mechanisms and involvement in human disease. Trends Genet, 18: 186-93.
Castelo-Branco P, Furger A, Wollerton M, Smith C, Moreira A and Proudfoot N. (2004). Polypyrimidine tract binding protein modulates efficiency of polyadenylation. Mol Cell Biol, 24: 4174-83.
Cho S, Kim J H, Back S H and Jang S K. (2005). Polypyrimidine tract-binding protein enhances the internal ribosomal entry site-dependent translation of p27Kip1 mRNA and modulates transition from G1 to S phase. Mol Cell Biol, 25: 1283-97.
Cornelis S, Tinton S A, Schepens B, Bruynooghe Y and Beyaert R. (2005). UNR translation can be driven by an IRES element that is negatively regulated by polypyrimidine tract binding protein. Nucleic Acids Res, 33: 3095-108.
Cote C A, Gautreau D, Denegre J M, Kress T L, Terry N A and Mowry K L. (1999). A Xenopus protein related to hnRNP I has a role in cytoplasmic RNA localization. Mol Cell, 4: 431-7.
De Marzo A M, Bradshaw C, Sauvageot J, Epstein J I and Miller G J. (1998). CD44 and CD44v6 downregulation in clinical prostatic carcinoma: relation to Gleason grade and cytoarchitecture. Prostate, 34: 162-8.
Deblandre G A, Marinx O P, Evans S S, Majjaj S, Leo O, Caput D, et al. (1995). Expression cloning of an interferon-inducible 17-kDa membrane protein implicated in the control of cell growth. J Biol Chem, 270: 23860-6.
Feltes C M, Kudo A, Blaschuk O and Byers S W. (2002). An alternatively spliced cadherin-11 enhances human breast cancer cell invasion. Cancer Res, 62: 6688-97.
Foy T M, Aruffo A, Bajorath J, Buhlmann J E and Noelle R J. (1996). Immune regulation by CD40 and its ligand GP39. Annu Rev Immunol, 14: 591-617.
Garcia-Blanco M A, Jamison S F and Sharp P A. (1989). Identification and purification of a 62,000-dalton protein that binds specifically to the polypyrimidine tract of introns. Genes Dev, 3: 1874-86.
Ghetti A, Pinol-Roma S, Michael W M, Morandi C and Dreyfuss G. (1992). hnRNP I, the polypyrimidine tract-binding protein: distinct nuclear localization and association with hnRNAs. Nucleic Acids Res, 20: 3671-8.
Gunthert U, Hofmann M, Rudy W, Reber S, Zoller M, Haussmann I, et al. (1991). A new variant of glycoprotein CD44 confers metastatic potential to rat carcinoma cells. Cell, 65: 13-24.
Hamilton B J, Genin A, Cron R Q and Rigby W F. (2003). Delineation of a novel pathway that regulates CD154 (CD40 ligand) expression. Mol Cell Biol, 23: 510-25.
He X, Ee P L, Coon J S and Beck W T. (2004). Alternative splicing of the multidrug resistance protein 1/ATP binding cassette transporter subfamily gene in ovarian cancer creates functional splice variants and is associated with increased expression of the splicing factors PTB and SRp20. Clin Cancer Res, 10: 4652-60.
Huang S, Deerinck T J, Ellisman M H and Spector D L. (1997). The dynamic organization of the perinucleolar compartment in the cell nucleus. J Cell Biol, 137: 965-74.
Jemal A, Murray T, Ward E, Samuels A, Tiwari R C, Ghafoor A, et al. (2005). Cancer statistics, 2005. CA Cancer J Clin, 55: 10-30.
Jin W, Bruno I G, Xie T X, Sanger L J and Cote G J. (2003). Polypyrimidine tract-binding protein down-regulates fibroblast growth factor receptor 1 alpha-exon inclusion. Cancer Res, 63: 6154-7.
Jin W, McCutcheon I E, Fuller G N, Huang E S and Cote G J. (2000). Fibroblast growth factor receptor-1 alpha-exon exclusion and polypyrimidine tract-binding protein in glioblastoma multiforme tumors. Cancer Res, 60: 1221-4.
Jin X, Burke W, Rothman K and Lin J. (2002). Resistance to p53-mediated growth suppression in human ovarian cancer cells retain endogenous wild-type p53. Anticancer Res, 22: 659-64.
Justesen J, Hartmann R and Kjeldgaard N O. (2000). Gene structure and function of the 2′-5′-oligoadenylate synthetase family. Cell Mol Life Sci, 57: 1593-612.
Kamath R V, Leary D J and Huang S. (2001). Nucleocytoplasmic shuttling of polypyrimidine tract-binding protein is uncoupled from RNA export. Mol Biol Cell, 12: 3808-20.
Kamath R V, Thor A D, Wang C, Edgerton S M, Slusarczyk A, Leary D J, et al. (2005). Perinucleolar compartment prevalence has an independent prognostic value for breast cancer. Cancer Res, 65: 246-53.
Kasper J S, Kuwabara H, Arai T, Ali S H and DeCaprio J A. (2005). Simian virus 40 large T antigen's association with the CUL7 SCF complex contributes to cellular transformation. J Virol, 79: 11685-92.
Knoch K P, Bergert H, Borgonovo B, Saeger H D, Altkruger A, Verkade P, et al. (2004). Polypyrimidine tract-binding protein promotes insulin secretory granule biogenesis. Nat Cell Biol, 6: 207-14.
Kosinski P A, Laughlin J, Singh K and Covey L R. (2003). A complex containing polypyrimidine tract-binding protein is involved in regulating the stability of CD40 ligand (CD154) mRNA. J Immunol, 170: 979-88.
Lou H, Gagel R F and Berget S M. (1996). An intron enhancer recognized by splicing factors activates polyadenylation. Genes Dev, 10: 208-19.
Lou H, Helfman D M, Gagel R F and Berget S M. (1999). Polypyrimidine tract-binding protein positively regulates inclusion of an alternative 3′-terminal exon. Mol Cell Biol, 19: 78-85.
Lukas J, Gao D Q, Keshmeshian M, Wen W H, Tsao-Wei D, Rosenberg S, et al. (2001). Alternative and aberrant messenger RNA splicing of the mdm2 oncogene in invasive breast cancer. Cancer Res, 61: 3212-9.
Matera A G, Frey M R, Margelot K and Wolin S L. (1995). A perinucleolar compartment contains several RNA polymerase III transcripts as well as the polypyrimidine tract-binding protein, hnRNP I. J Cell Biol, 129: 1181-93.
Mitchell S A, Brown E C, Coldwell M J, Jackson R J and Willis A E. (2001). Protein factor requirements of the Apaf-1 internal ribosome entry segment: roles of polypyrimidine tract binding protein and upstream of N-ras. Mol Cell Biol, 21: 3364-74.
Moreira A, Takagaki Y, Brackenridge S, Wollerton M, Manley J L and Proudfoot N J. (1998). The upstream sequence element of the C2 complement poly(A) signal activates mRNA 3′ end formation by two distinct mechanisms. Genes Dev, 12: 2522-34.
Oberstrass F C, Auweter S D, Erat M, Hargous Y, Henning A, Wenter P, et al. (2005). Structure of PTB bound to RNA: specific binding and implications for splicing regulation. Science, 309: 2054-7.
Ozols R F, Bookman M A, Connolly D C, Daly M B, Godwin A K, Schilder R J, et al. (2004). Focus on epithelial ovarian cancer. Cancer Cell, 5: 19-24.
Persengiev S P, Zhu X and Green M R. (2004). Nonspecific, concentration-dependent stimulation and repression of mammalian gene expression by small interfering RNAs (siRNAs). Rna, 10: 12-8.

Sanchez Lockhart M, Hajos S E, Basilio F M, Mongini C and Alvarez E. (2001). Splice variant expression of CD44 in patients with breast and ovarian cancer. Oncol Rep, 8: 145-51.
Scacheri P C, Rozenblatt-Rosen O, Caplen N J, Wolfsberg T G, Umayam L, Lee J C, et al. (2004). Short interfering RNAs can induce unexpected and divergent changes in the levels of untargeted proteins in mammalian cells. Proc Natl Acad Sci USA, 101: 1892-7.
Schroder W, Rudlowski C, Biesterfeld S, Knobloch C, Hauptmann S and Rath W. (1999). Expression of CD44(v5-10) splicing variants in primary ovarian cancer and lymph node metastases. Anticancer Res, 19: 3901-6.
Silberstein G B, Van Horn K, Strickland P, Roberts C T, Jr. and Daniel C W. (1997). Altered expression of the WT1 wilms tumor suppressor gene in human breast cancer. Proc Natl Acad Sci USA, 94: 8132-7.
Stickeler E, Kittrell F, Medina D and Berget S M. (1999). Stage-specific changes in SR splicing factors and alternative splicing in mammary tumorigenesis. Oncogene, 18: 3574-82.
Stoilov P, Meshorer E, Gencheva M, Glick D, Soreq H and Stamm S. (2002). Defects in Pre-mRNA Processing as Causes of and Predisposition to Diseases. DNA Cell Biol, 21: 803-18.
Suzuki F, Arase Y, Suzuki Y, Tsubota A, Akuta N, Hosaka T, et al. (2004). Single nucleotide polymorphism of the MxA gene promoter influences the response to interferon monotherapy in patients with hepatitis C viral infection. J Viral Hepat, 11: 271-6.
Wagner E J and Garcia-Blanco M A. (2001). Polypyrimidine tract binding protein antagonizes exon definition. Mol Cell Biol, 21: 3281-8.
Wang J and Pederson T. (1990). A 62,000 molecular weight spliceosome protein crosslinks to the intron polypyrimidine tract. Nucleic Acids Res, 18: 5995-6001.
Wang Z, Lo H S, Yang H, Gere S, Hu Y, Buetow K H, et al. (2003). Computational analysis and experimental validation of tumor-associated alternative RNA splicing in human cancer. Cancer Res, 63: 655-7.
Wielenga V J, Heider K L H, Offerhaus G J, Adolf G R, van den Berg F M, Ponta H, et al. (1993). Expression of CD44 variant proteins in human colorectal cancer is related to tumor progression. Cancer Res, 53: 4754-6.
Wiznerowicz M and Trono D. (2003). Conditional suppression of cellular genes: lentivirus vector-mediated drug-inducible RNA interference. J Virol, 77: 8957-61.
Wollerton M C, Gooding C, Robinson F, Brown E C, Jackson R J and Smith C W. (2001). Differential alternative splicing activity of isoforms of polypyrimidine tract binding protein (PTB). Rna, 7: 819-32.
Wollerton M C, Gooding C, Wagner E J, Garcia-Blanco M A and Smith C W. (2004). Autoregulation of polypyrimidine tract binding protein by alternative splicing leading to nonsense-mediated decay. Mol Cell, 13: 91-100.
Xie J, Lee J A, Kress T L, Mowry K L and Black D L. (2003). Protein kinase A phosphorylation modulates transport of the polypyrimidine tract-binding protein. Proc Natl Acad Sci USA, 100: 8776-81.
Xu Q and Lee C. (2003). Discovery of novel splice forms and functional analysis of cancer-specific alternative splicing in human expressed sequences. Nucleic Acids Res, 31: 5635-43.
Zang W Q, Li B, Huang P Y, Lai M M and Yen T S. (2001). Role of polypyrimidine tract binding protein in the function of the hepatitis B virus posttranscriptional regulatory element. J Virol, 75: 10779-86.

We claim:

1. A method of diagnosing cancer in a mammalian subject comprising measuring the level of polypyrimidine-tract binding protein (PTB) in a tissue sample obtained from the subject, wherein an elevated level of PTB in the sample is indicative the subject is positively diagnosed with cancer.

2. The method of claim 1, wherein the subject is a human subject.

3. The method of claim 1, wherein the cancer is ovarian cancer.

4. The method of claim 1, wherein the cancer is breast cancer.

5. A method of inhibiting proliferation of a mammalian cancer cell comprising suppressing the level of polypyrimidine-tract binding protein (PTB) in the cell.

6. The method of claim 5, wherein the mammalian cell is a human cell.

7. The method of claim 5, wherein the cancer cell is an ovarian cancer cell.

8. The method of claim 5, wherein the cancer cell is a breast cancer cell.

9. The method of claim 5, wherein the suppression of the level of PTB is accomplished by providing the cancer cell with an siRNA targeting to PTB mRNA.
